(19) United States
(12) Patent Application Publication
(10) Pub. No.: US 2005/0079187 A1
(43) Pub. Date: Apr. 14, 2005

(54) Composition, kit and method for reducing plaque formation, tooth decay and incidence of caries

(76) Inventor: Wallace J. Gardner, Cambridge, MA (US)

Correspondence Address: NIELDS & LEMACK, 176 EAST MAIN STREET, SUITE 7, WESTBORO, MA, 01581, US

(21) Appl. No.: 10/685,058

(22) Filed: Oct. 14, 2003

(51) Int. Cl. A61K 9/00; A61K 31/65; A61K 7/16

(52) U.S. Cl. 424/400; 514/152; 424/49

(57) ABSTRACT

Therapeutic composition and method effective for reducing plaque formation and reducing tooth decay. The therapeutic composition is a formulation comprising an antibiotic, preferably a tetracycline, most preferably doxycycline, which optionally has not been chemically modified to eliminate antimicrobial efficacy. The antibiotic is preferably in a liquid vehicle, preferably one that contains at least 35%, most preferably 39% alcohol by volume. The teeth of a mammal are brushed with the therapeutic composition, preferably multiple times daily.

### BACKGROUND OF THE INVENTION

The accumulation of bacteria in the oral cavity of mammals, such as on the teeth or tongue or in the tissue area between the teeth and gums can contribute to plaque formation, tooth decay and periodontal disease. Treatment of the oral cavity with antibiotics to reduce or eliminate the effects of microorganisms is known. For example, broad spectrum antibiotics such as tetracyclines and metronidazole have been used in the treatment of periodontal disease to reduce oral cavity microflora. Typically such use has been systemic, which can result in various undesirable side effects, including the threat or danger of building allergies or immunity to the antibiotic, overgrowth of opportunistic yeast and fungi and intestinal disturbances.

Many common inflammatory diseases, such as sinusitis, diseases of the gastrointestinal tract (including those that manifest themselves in stomach and bowel problems), the common cold, influenza, allergies, halitosis, pneumonia, etc., also may be caused by viruses and/or microorganisms. Often the source of the microorganisms and viruses is the sinuses, typically via the oral cavity, especially the ear, nose and throat passages. Once the microorganisms and/or viruses are resident in the oral cavities or sinuses (e.g., the maxillary, frontal, ethmoid and ophenoidal), they can continually cause inflammation and infection through circulation in the blood stream.

It would be desirable to provide a therapeutic composition and kit that retards the production of plaque on the teeth, reduces or eliminates tooth decay, particularly the incidence of caries, and that does not suffer from the adverse side effects of existing medications.

It further would be desirable to provide a method of brushing teeth that effectively retards the production of plaque on the teeth, reduces or eliminates tooth decay, particularly the incidence of caries, and that does not suffer from the adverse side effects of existing medications.

### SUMMARY OF THE INVENTION

The problems of the prior art have been overcome by the present invention, which provides a therapeutic composition and kit having plaque reduction activity, reduces or eliminates tooth decay, as well as a method of brushing teeth with the composition to retard plaque formation and reduce or eliminate caries. In a preferred embodiment, the therapeutic composition is a formulation comprising an antibiotic, preferably a tetracycline, most preferably doxycycline, which has not been chemically modified to eliminate antimicrobial efficacy. The antibiotic is preferably in a liquid vehicle, preferably one that contains at least 35%, most preferably at least 39%, alcohol by volume. The therapeutic composition is preferably in local delivery form and is preferably self-administered by brushing the teeth. Gargling can be carried out in addition. The therapeutic composition most preferably is a self-delivered formulation in local delivery form that consists essentially of a tetracycline, most preferably doxycycline, which has not been chemically modified to eliminate antimicrobial efficacy, and a liquid vehicle, more preferably one which contains at least 35%, preferably at least 39% alcohol by volume, and most preferably one which consists essentially of sterile water or a rinse as defined below or the like, which tetracycline is preferably present in the formulation in the amount of between 50 to 100 mgs per ounce of liquid vehicle.

A further embodiment of the present invention relates to a preparation that includes a kit comprising the therapeutic compositions mentioned above along with instructions for use for reducing plaque formation, and/or reducing or eliminating tooth decay, particularly the incidence of caries.

### DETAILED DESCRIPTION OF THE INVENTION

The therapeutic compositions of the present invention are local delivery compositions that have activity against tooth decay, and comprise an antibiotic, preferably a tetracycline that optionally has not been chemically modified to eliminate antimicrobial efficacy. The compositions are effective to prevent pollutants from remaining on or near the teeth and in the tissue between the teeth and gums in mammals, especially humans.

The tetracyclines useful in the compositions and method of the present invention include apicycline, chlortetracycline, clomocycline, demeclocycline, doxycycline, guamecycline, lymecycline, meclocycline, methacycline, minocycline, oxytetracycline, penimepicycline, pipacycline, rolitetracycline, sancycline, senociclin, and tetracycline. Doxycycline is particularly preferred.

The therapeutic composition of the invention also includes a liquid carrier that is any compatible, nontoxic substance suitable to deliver the antibiotic. Carriers can include sterile water, sodium chloride (e.g. 0.9%), alcohol, and mixtures thereof. Water is a preferred liquid for the liquid vehicle and the vehicle even more preferably comprises at least about 35% alcohol by volume, more preferably 35-40% alcohol and most preferably about 39% alcohol. Preferably, the therapeutic composition does not include a morpholinoamino alcohol or pharmaceutically-acceptable salt thereof. One particularly preferred formulation includes a mixture of doxycycline with 0.064% thymol, 0.092% eucalyptol, 0.060% methylsalicylate, 0.042% menthol, 39% alcohol, poloxamer 407, water, sodium benzoate and caramel. Other suitable formulations include a mixture of doxycycline with commercially available mouthwashes, oral rinses or anti-plaque formulations. Pharmaceutically acceptable adjuvants may also be incorporated into the therapeutic composition. In certain therapeutic compositions of the invention, antiphlogisitic agents are not preferred. The compositions of the present invention also may contain various inactive ingredients, such as sweeteners (natural and artificial), flavorings (natural or artificial), colorants, preservatives, etc.

The amount of antibiotic used in the formulation depends upon the frequency of the dose, the particular infection or malady and the severity of the infection or malady being treated or prevented, the method of local administration of the composition and the effect desired (including whether the treatment is a prophylactic or therapeutic application). Actual methods for preparing administrable compositions will be known or apparent to those skilled in the art and are described in more detail in, for example, Remington's Pharmaceutical Science, 17^{th} Ed., Mack Publishing Co., Easton, Pa. (1985), which is incorporated herein by reference. Preferably the formulation contains from about 50 to about 100 mgs tetracycline, preferably doxycycline, per ounce of total liquid in the formulation. The formulation is prepared by mixing the tetracycline with the liquid vehicle and shaking the mixture.

The compositions also can be provided in the form of a kit, including the tetracycline and the liquid vehicle, optionally together with instructions for use in brushing the teeth of a mammal and optionally gargling. The instructions preferably include suitable dosage amounts, as well as frequency of use guidelines.

In the preferred form of the present invention, the therapeutic composition is a liquid composition into which the toothbrush is dipped. For example, the preferably the toothbrush is dipped into the solution multiple times during the tooth brushing procedure, most preferably one time for each quadrant of the mouth. The teeth are then brushed in the normal manner after each dipping. Those skilled in the art will appreciate that the toothbrush can be dipped more than once for each quadrant of the mouth if desired. Preferably the teeth are brushed at least twice daily, most preferably in the morning and evening and after each meal. Although the present inventor is not to be limited by any particular theory, it is believed that the action of the toothbrush against the tissue alongside the teeth allows the composition of the invention to penetrate and kill microorganisms.

In addition to brushing the teeth, gargling with the present composition can be carried out before, during or after the tooth brushing procedure. Preferably the gargling includes introducing the composition into the mouth and holding it in the mouth for at least 30 seconds and preferably at least 1 minute. During the holding period, the composition is preferably swirled around the mouth in order to bathe the teeth and gums in the composition. The composition is then expelled from the mouth into a waste receptacle.

The amount of antibiotic, e.g. doxycycline, used in the compositions and the dosage selection is an amount and dosage effective to achieve plaque reduction and/or decay reduction, i.e. the amount necessary to effectively treat the bacteria/microorganisms that contribute to the creation of plaque and decay. Although suitable amounts of antibiotic in the therapeutic composition are within the range of 50 to 100 mg per ounce of liquid vehicle, those skilled in the art will determine optimum concentrations and dosages from clinical experience in order to carry out the method of the invention.

Suitable mammals include mammals having teeth, particularly humans and domestic animals, such as dogs and cats.

### EXAMPLE 1

A 100 mg and a 50 mg capsule of doxycycline are opened and the contents added to 3 ounces of antiseptic mouthwash. This mixture is shaken until the doxycycline is dispersed in the mouthwash.

A patient dips his toothbrush in the solution and brushes the teeth in one quadrant of the mouth. This procedure is repeated for each quadrant. One third ounce of the solution is then introduced into the mouth and swirled in the mouth for 30 seconds and gargled.

1. A therapeutic composition having plaque formation inhibiting activity, comprising a plaque formation inhibiting effective amount of a tetracycline in a liquid vehicle.

2. The therapeutic composition of claim 1, wherein said tetracycline has not been chemically modified to eliminate antimicrobial efficacy.

3. The therapeutic composition of claim 1, wherein said therapeutic composition is devoid of a morpholinoamino alcohol or pharmaceutically acceptable salt thereof.

4. The therapeutic composition of claim 1, wherein said liquid vehicle comprises at least 35% alcohol by volume.

5. The composition of claim 4, wherein said liquid vehicle further comprises a mixture of two or more of thymol, menthol, eucalyptol and methylsalicylate.

6. The composition of claim 1, wherein said tetracycline is doxycycline.

7. A method of reducing plaque formation on teeth of a mammal, comprising brushing said teeth a composition comprising a plaque formation inhibiting effective amount of a tetracycline in a liquid vehicle.

8. The method of claim 7, wherein said mammal is a human.

9. The method of claim 7, wherein said tetracycline is doxycycline.

10. A method of reducing tooth decay in a mammal, comprising brushing said teeth with a composition comprising a tooth decay inhibiting effective amount of a tetracycline in a liquid vehicle.

11. The method of claim 10, wherein said mammal is a human.

12. The method of claim 10, wherein said tetracycline is doxycycline.

13. A kit, comprising a tetracycline, a liquid vehicle for said tetracycline, and instructions for use of said tetracycline and liquid vehicle for brushing teeth with said tetracycline and liquid vehicle.

14. The kit of claim 13, wherein said tetracycline is doxycycline.
